(19) United States
(12) Patent Application Publication
(10) Pub. No.: US 2024/0350065 A1
(43) Pub. Date: Oct. 24, 2024

(54) METHOD FOR DETECTING A CARDIAC ISOLATION STATUS OF A MEASUREMENT LOCATION IN THE PRESENCE OF FAR FIELD INTERFERENCE

(71) Applicant: CathVision ApS, Kopenhagen (DK)

(72) Inventors: Rune PAAMAND, Humlebæk (DK); Mads Emil MATTHIESEN, Genarp (SE)

(21) Appl. No.: 18/684,800

(22) PCT Filed: Aug. 3, 2022
(86) PCT No.: PCT/EP2022/071843

§ 371 Date: Feb. 19, 2024

(30) Foreign Application Priority Data
Aug. 20, 2021 (EP) 21192435.2

(51) Int. Cl. A61B 5/308; A61B 5/00; A61B 5/287; A61B 5/346

(52) U.S. Cl. CPC A61B 5/308; A61B 5/287; A61B 5/346; A61B 5/7217; A61B 5/725; A61B 5/7264

(57) ABSTRACT

The invention concerns a method for determining a cardiac isolation status of a measurement location (1) in the presence of far field interference by analysing a multi-channel intracardiac electrogram (2) of the measurement location (1) via a control system (3), wherein in an identification routine (9) the control system (3) applies an activation search algorithm to analysis windows (10) of at least 400 ms in at least two different channels (11) of the intracardiac electrogram (2), wherein the activation search algorithm identifies windows (W) of local activation potentials (12) inside of the analysis windows (10), wherein in a classification routine (15) the control system (3) analyses the local activation potentials (12) to determine the cardiac isolation status of the measurement location (1).

### CROSS-REFERENCE TO RELATED APPLICATIONS

The present application is a U.S. national stage of International Application No. PCT/EP2022/071843, filed Aug. 3, 2022, and claims priority to European Patent Application No. EP 21192435.2, filed Aug. 20, 2021.

### BACKGROUND OF INVENTION

The invention relates to a method for determining a cardiac isolation status of a measurement location in the presence of far field interference and to a control system adapted to execute the method.

The present method is particularly concerned with atrial fibrillation and atrial flutter. Electrically, atrial fibrillation is chaotic activation of muscle cells of the atrial. During atrial fibrillation, the atria only minimally contribute to the function of the heart. Atrial fibrillation, therefore, reduces the output of the heart but is not imminently dangerous. However, when becoming chronical, atrial fibrillation is correlated width increased morbidity and mortality. One treatment option for atrial fibrillation is ablation therapy. Ablation is the destruction of the cells that allow electrical wave re-entry to reduce chaotic activation of the atrial muscle cells.

A recommended treatment for atrial fibrillation includes pulmonary vein isolation. Pulmonary vein isolation can be performed with various ablation techniques, including radio frequency ablation, cryo balloon ablation and pulsed-field ablation. While these techniques apply energy differently, their common endpoint is the isolation of the electrical activity of the pulmonary veins from the rest of the atrium.

Generally, ablation therapy is successful, if the targeted location is electrically isolated from the rest of the heart. There are different methods for assessing the success of the ablation therapy. These include costly 3D mapping of ablation points and measurements of force or temperature during ablation and electrogram analysis.

One known method (EP 3 139 828 B1) focusses on a morphology analysis of local activation potentials of a measurement location. In this method, the local activation potentials are classified into groups according to the number and characteristics of their peaks. An analysis of the distribution of the potentials across the morphological groups allows a good classification of a cardiac isolation status of the measurement location. A method based on electrical signals is cheap in the implementation and easily usable. Further, measuring electrical waves targets the actual physical effect underlying atrial fibrillation rather than a secondary factor.

The known method provides good results. However, in the known method the local activation potentials of the measurement location are detected based on the detection of a reference CS-potential. This CS-potential is detected by a coronary sinus catheter. Based on this reference CS-potential, a predetermined window of approximately 200 ms is chosen. This window is defined as containing the local activation potentials of the channels related to the measurement location. Therefore, the reliability of the known method depends on the patient being in a sinus rhythm.

There exists a demand for a method for determining an isolation status using electrograms and analysis of local activation potentials usable without a-priori knowledge about the location of the local activation potentials from a reference potential. This is necessary for example if a cardiac isolation status of a patient should be determined while the patient is in atrial fibrillation. During atrial fibrillation, the time relation between the CS-potential and local activations may be broken. While the possibility to cardiovert a patient to a normal sinus rhythm exists, this may not always be desired.

It is further problematic that cardiac electrograms contain not only local activation potentials but also far field interference. Measurement of the CS-potential also typically requires a separate CS-catheter in addition to a catheter that may be located in the pulmonary vein. It is desirable to identify methods for determining cardiac isolation status that can also be performed with a single catheter.

It is therefore an object of the present invention to provide a method for detecting a cardiac isolation status in the presence of far field interference usable without being able to rely on a reference CS-potential for detecting local activations.

The above-noted object is solved by the methods disclosed herein.

### BRIEF SUMMARY OF THE INVENTION

The main realization of the present invention is that by analyzing multiple channels of an intracardiac electrogram independently and searching for local activation potentials on the channels without using a single resulting time window on all channels, sufficient actual local activation potentials can be found even during atrial fibrillation to enable analyzing those local activation potentials to determine a cardiac isolation status of the measurement location.

In particular, this enables determining a cardiac isolation status of a measurement location during atrial fibrillation.

In detail, a method for determining a cardiac isolation status of a measurement location in the presence of far field interference by analyzing a multi-channel intracardiac electrogram of the measurement location via a control system, wherein in an identification routine the control system applies an activation search algorithm to analysis windows of at least 400 ms in at least two different channels of the intracardiac electrogram, wherein the activation search algorithm identifies windows of local activation potentials inside of the analysis windows, wherein in a classification routine the control system analyzes the local activation potentials to determine the cardiac isolation status of the measurement location, is proposed.

Very preferred applications of the proposed method are disclosed herein. In particular during atrial fibrillation or atrial flutter, local activation potentials, particularly pulmonary vein local activation potentials, cannot be detected by relying on a-priori-knowledge about their position in time from a reference CS-potential.

In an embodiment disclosed herein, the control system analyses in the classification routine a morphology of the local activation potentials to determine a cardiac isolation status. It has been realized that a morphology classification of local activation potentials can be done effectively even during atrial fibrillation. In general, morphologies with less fractionated peaks, lower peak frequencies, lower peak sharpness and peaks of lesser amplitudes indicate a successful isolation. Different preferred morphology groups are disclosed that have been shown to enable a successful isolation status determination.

Other embodiments disclosed herein relate to preferred widths of the analysis windows chosen to ensure mostly having at least one physiological local activation potential in each analysis window. It is preferred to choose the width of the analysis window such that at least one physiological local activation potential is present in each analysis window. This allows adapting the activation search algorithm such that it can always find a local activation potential because the analysis window contains at least one. For the present algorithm it is not necessary to find all local activation potentials. Nevertheless, in an embodiment, sliding windows are used to find multiple local activation potentials per channel.

Another embodiment is concerned with the possibility of using the proposed method to verify the success of an ablation procedure. As an ablation procedure is an invasive surgical operation, observing a patient for days to determine the success of an ablation procedure is not preferred. Rather, the success of the ablation procedure should be determined during the intervention.

The activation search algorithm may comprise a peak detection algorithm described herein.

The control system may identify a fixed number of local activation potentials per channel. This number may be smaller or equal to the number of physiologically present local activation potentials. In this way the number of incorrectly identified local activation potentials can be reduced.

In a preferred embodiment, the control system may execute an interference signal removal step prior to the identification step, in particular to remove far field interference signals. The specification and claims disclose removal of signals around and/or relative to pacing artifacts, CS-potentials and ECG-waves. By removing time windows related to sources of far field interference, the chance of detecting actual local activation potentials is greatly increased. This removal is called blanking. Such blanking may also be done in a weighted manner to enable identifying local activation potentials that partly overlap with the blanked time frames. The blanking may for example be weighted by a gauss curve fully or almost fully removing the electrogram in the middle of the time window to be blanked and only reducing an amplitude at the edges. Further details of the weighted blanking are disclosed herein.

To reduce a number of incorrectly identified local activation potentials, the control system may execute a quality control step. Sections of the electrogram or even channels of the electrogram may be removed from consideration before or after the identification step based on a quality parameter.

The method also relates to preferably used catheters.

Another teaching disclosed herein, which is of equal importance, is directed to a control system configured to perform the proposed method. All explanations given with respect to the proposed method are fully applicable.

### BRIEF DESCRIPTION OF THE DRAWINGS

In the following, an embodiment of the invention is explained with respect to the drawings. The drawings show in

FIG. 1 the proposed control system during the measurement of a multi-channel intracardiac electrogram, and in

FIG. 2 the proposed method in an exemplary application.

### DETAILED DESCRIPTION OF THE INVENTION

The proposed method is used for determining a cardiac isolation status of a measurement location 1 in the presence of far field interference by analyzing a multi-channel intracardiac electrogram 2 of the measurement location 1 via a control system 3. The cardiac isolation status may be a binary yes/no decision or a percentage of a probability of isolation of the measurement location 1. The multi-channel intracardiac electrogram 2 is preferably measured by an intracardiac catheter 4 comprising a number of electrodes 5, for example ten electrodes 5. These electrodes 5 have a certain spatial extension. Therefore, the measurement location 1 also has a certain spatial extension. It is not necessary however for all electrodes 5 of the measurement catheter 6 to be located at the measurement location 1. It might be the case that the measurement catheter 6 is placed across a boundary of an ablation lesion 7 for example.

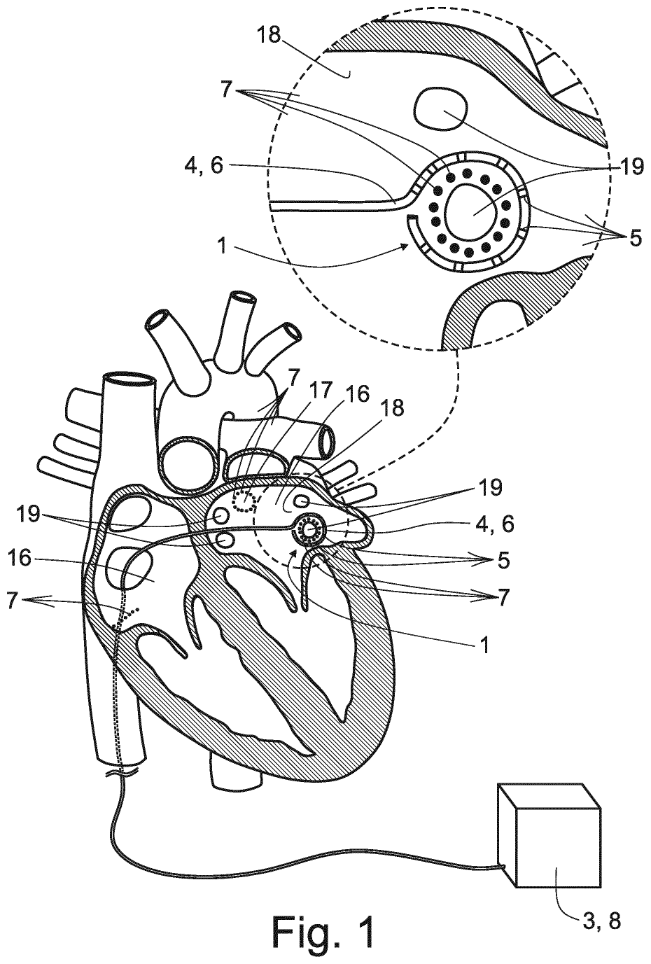

The control system 3 may be a local unit 8 with a processor, possibly a user interface and the like as shown in FIG. 1. It may also comprise in one embodiment a cloud processor. It this therefore not necessary for the control system 3 to be confined to a single device.

In an identification routine 9 the control system 3 applies an activation search algorithm to analysis windows 10 of at least 400 ms in at least two different channels 11 of the intracardiac electrogram 2. A channel 11 is defined as the measurement between at least two electrodes 5. The proposed method serves to determine a cardiac isolation status when local activation potentials 12 cannot be reliably located relative to a reference CS-potential 13. This is particularly true during atrial fibrillation.

The activation search algorithm identifies windows W of local activation potentials 12 inside of the analysis windows 10. It is essential that these windows W of local activation potentials 12 can differ regarding their location in time between channels 11 and are not set equal for all channels 11 relative to the timing of a reference potential 14. Therefore, the search field for these windows W is chosen to be at least 400 ms wide. In general, the windows W of the local activation potentials 12 should comprise all or most of the relevant waveform of the respective local activation potential 12. The activation search algorithm may be an algorithm searching directly for the window W or searching for a single point in time assigned to the local activation potential 12 and derive the window W from there.

By searching local activation potentials 12 without a reference timing, it becomes possible to determine a cardiac isolation status without knowledge about any reference timing.

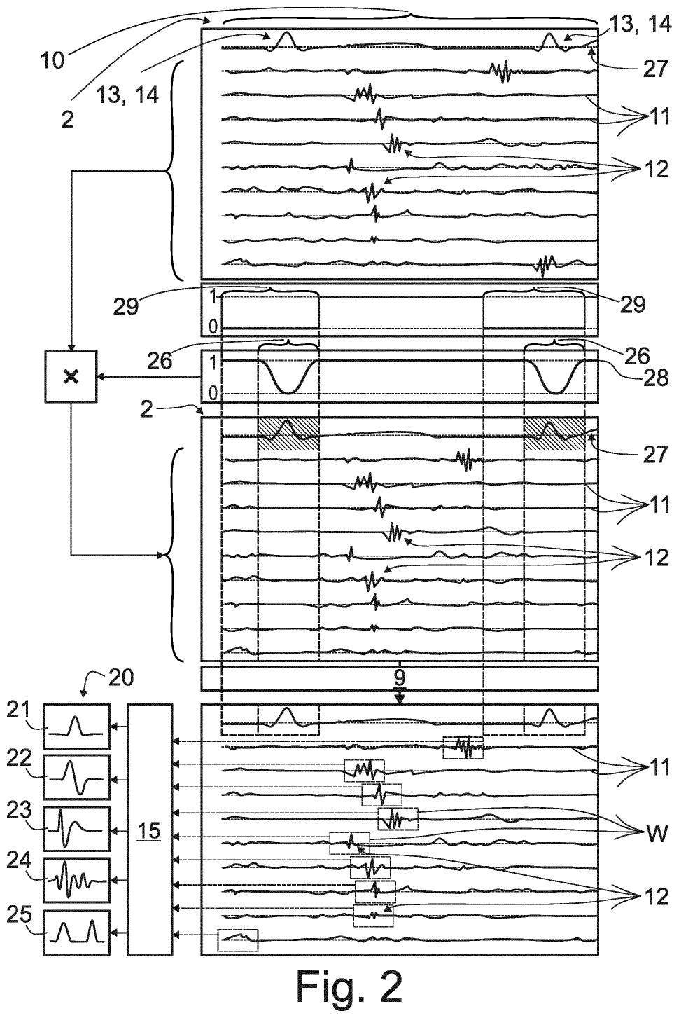

FIG. 2 shows the application of the identification routine 9 to a multi-channel intracardiac electrogram 2 and resulting windows W of local activation potentials 12. Here and preferably the windows W of the local activation potentials 12 have a width of at most 250 ms, preferably at most 200 ms, more preferably at most 175 ms and more preferably at most 150 ms. Here they have a width of approximately 128 ms. Preferably, the width of the windows W of local activation potentials 12 is at least 50 ms.

In a classification routine 15 the control system 3 analyses the local activation potential 12 to determine the cardiac isolation status of the measurement location 1. The classification routine 15 may use the algorithm of EP 3 139 828 B1, which is hereby included by reference.

As has already been described, it is preferred that the intracardiac electrogram 2 has been recorded during an atrial arrhythmia, in particular atrial fibrillation or atrial flutter. The proposed method allows determining the cardiac isolation status even in those cases.

As is schematically shown in FIG. 1, the measurement location 1 may be at least partly located inside an atrium 16, in particular inside the left atrium 16. The measurement location 1 may be an isle 17 in the wall 18 of the left atrium 16 or at the entrance of a pulmonary vein 19. There are different locations that can be targeted with ablation. A main target are the entrances of the pulmonary veins 19 into the left atrium 16. Successfully isolating the pulmonary veins 19 may be the target of an ablation therapy. Therefore the measurement location 1 may be at least partly located inside a pulmonary vein 19.

In general, the proposed method can be used prior to, during and/or after an ablation therapy. It can be used to determine an isolation status of a potential target for ablation in which case the isolation status may comprise information about how the measurement location 1 is involved with the conduction of electrical activations. Here and preferably the cardiac isolation status contains information about how well an ablation therapy has isolated the measurement location 1 from a remainder of the heart. Here and preferably the proposed method is used during and or after an ablation therapy. The measurement location 1 may then be the target of the ongoing or concluded ablation therapy.

Turning now to the classification routine 15, it may be the case that in the classification routine 15 the control system 3 analyses a morphology of the local activation potentials 12 to determine the cardiac isolation status of the measurement location 1. It has been found that information about the isolation status is contained in the complexity and amplitude of the local activation potentials 12. The morphology of the local activation potentials 12 depends on whether these are caused by propagation of a more global activation of the heart or a local pacing event and whether the general electrical situation is chaotic as in atrial fibrillation.

In the classification routine 15 the control system 3 may classify the local activation potential 12 into morphology groups 20 and preferably determines based on the distribution of local activation potentials 12 across the groups the cardiac isolation status. It is particularly advantageous that incorrectly identified local activation potentials 12, which may be caused by noise or far field interference, often show a morphology distinct from the morphology of local activation potentials 12 from a non-isolated location or an isolated location, allowing to ignore these false detections.

The control system 3 may classify local activation potentials 12 into morphology groups 20 based on a number of characteristic peaks of the local activation potential 12. For this, not every plateau in the electrogram 2 necessarily counts as a characteristic peak. Preferably, the control system 3 classifies a peak with at least a predetermined amplitude and/or with at least a predetermined slope and/or with at most a predetermined slope and/or with at least a predetermined minimum peak distance and/or with at most a predetermined maximum peak distance and/or based on a peak morphology, in particular a minimum and/or maximum peak angle, as a characteristic peak. Classification as a characteristic peak can also be based on the peaks having a predetermined peak frequency and/or a predetermined peak sharpness.

Here and preferably the morphology groups 20 comprise a group for local activation potentials 12 with a single characteristic peak, “monophasic 21”, and/or a group for local activation potentials 12 with exactly two characteristic peaks, “biphasic 22” and/or exactly three characteristic peaks, “triphasic 23”, and/or more than three characteristic peaks, “multiphasic 24”. The morphology groups 20 may further or alternatively comprise a group for local activation potentials 12 with at least two characteristic peaks separated by a predetermined time, “double potentials 25”. They may further comprise a group for local activation potentials 12 that show a morphology associated with erroneously detected local activation potentials 12 either stemming from noise or far field interference for example.

It is preferred to choose the analysis windows 10 such that at least one physiological local activation potential 12 is probably or surely present. For this, the analysis windows 10 may have a width of at least 400 ms, preferably at least 800 ms, more preferably at least 1.25 s. In a preferred embodiment, the analysis windows 10 may have a width of at most 3 s, preferably at most 2 s, more preferably at most 1.75 s. Here the analysis windows 10 have a width of 1.5 s.

It is preferred to extract multiple local activation potentials 12 from each channel 11 to enable a good statistical analysis of the local activation potentials 12, in particular of their morphology groups 20. Here and preferably in total at least 2, preferably at least 5, in particular at most 10, local activation potentials 12 are extracted. To extract multiple local activation potentials 12 per channel, the analysis windows 10 may be overlapping or non-overlapping sliding windows over a measurement time for each channel. Preferably at least two local activation potentials 12 are extracted per channel 11 whereby in particular at least one local activation potential 12 may be extracted per analysis window 10. The measurement time may be chosen to be at least 1 s, preferably at least 2.5 s, more preferably at least 5 s. The measurement time may be chosen to be at most 10 s.

As mentioned above, the control system 3 may carry out the identification routine 9 and the classification routine 15 on a multi-channel intracardiac electrogram 2 of the measurement location 1 recorded after an ablation procedure applied near to, in particular around, the measurement location 1 to determine the cardiac isolation status of the measurement location 1. The control system 3 may thereby evaluate the success of the ablation procedure.

It may be preferred to compare data before and after the ablation procedure. Therefore, it may be the case that additionally the control system 3 carries out at least the identification routine 9 on a multi-channel intracardiac electrogram 2 of the measurement location 1 recorded prior to the ablation procedure and determines the cardiac isolation status based on a comparison of the local activation potentials 12 prior to and after the ablation procedure. The measurement locations 1 prior to and after the ablation procedure do not have to be identical, a reasonable co-incidence may suffice.

The activation search algorithm may comprise a peak detection algorithm for finding the local activation potentials 12 and subsequently the windows W of the local activation potentials 12. The peak detection algorithm may be based on non-linear filters, in particular wavelet filters, and/or it may be based on a transformation of the electrogram 2, in particular a wavelet transformation. The peak detection algorithm may comprise a peak detection by an amplitude. For example, the highest amplitude inside of a given time frame may be detected as a peak.

Returning to the detection of local activation potentials 12 by taking into consideration their frequency under physiological conditions, in the identification routine 9 the control system 3 may identify a fixed number of local activation potentials 12 per channel 11 and/or per analysis window 10. Preferably the control system 3 in the identification routine 9 may identify a fixed number of local activation potentials 12 per time interval. This time interval may be the measurement window. The fixed number may be based on a physiological and/or measured heartrate. It may be the case that the fixed number is a maximum of one local activation potential 12 per at least 500 ms, preferably per at least 800 ms, more preferably per at least 1 s. By detecting only a single local activation potential 12 inside of any given time window, for example 800 ms, it is possible to ensure that the activation search algorithm always has the possibility of finding a real local activation potential 12.

With regards to FIG. 2 the removal of interference signals, in particular from far field interference, will now be described in more detail. The control system 3 may execute an interference signal removal step prior to the identification step. The interference signal removal step may comprise complete or weighted blanking of time intervals 26 around and/or relative to pacing artefacts and or CS-potentials 13 and or ECG-waves 27. ECG-waves 27 may comprise P-waves, QRS-complexes and/or T-waves, that can be associated with atrial and ventricular activity. Generally, other artefacts may be blanked too. This blanking in the weighted form is shown in FIG. 2.

In a simple embodiment, the blanking can be seen as removing part of the electrogram 2 or multiplying the respective part of the electrogram 2 with zero. Here and preferably the blanking is applied to all channels 11. In the weighted form, the blanked time interval 26 of the electrogram 2 is not completely multiplied with zero. It may be only reduced in amplitude, in particular at the edges and near the edges of the time interval 26, where the far field interference is expected to be moderate. This makes it possible to find local activation potentials 12 partly overlapping with the blanked time interval 26.

The pacing artefacts and/or the CS-potentials 13 and or the ECG-waves 27 may be detected on an electrogram 2 different from the multi-channel intracardiac electrogram 2, the electrogram 2 be a coronary sinus or a surface electrogram 2. These are generally more suited to identify for example CS-potentials 13 and ECG-waves 27, such as P-waves, QRS-complexes and T-waves, associated with the atrial and ventricular activity, than a potentially isolated pulmonary vein 19 electrogram 2.

The CS-potentials 13 and ECG-waves 27 may be detected by known algorithms, for example by using peak detection on the surface electrogram 2. The pacing artefacts can be detected through peak amplitude detection and/or slope analysis.

As part of the interference signal removal step or as an independent step, a classification of interference signals may be done and may be used for blanking said interference signals. Here and preferably, the interference signals are classified as at least one of pacing artifacts and/or far field interference and/or instable activation potentials and/or noisy activation potentials. Detected interference signals may be excluded from being identified as local activation potentials 12.

Pacing artifacts are signals generated by methods of cardiac pacing. The pacing artifacts may be classified by analyzing voltage characteristics, in particular a slope of the channels 11. Local activation potentials 12 show a physiological delay between channels 11 respectively the real locations associated with the channels. Therefore, the control system 3 may classify signals that occur inside of a predetermined short time window within many or all channels 11 as pacing artifacts. Alternatively, the control system 3 may comprise an input for external pacing timing.

The far field interference may be classified by determining the timing of the QRS activations from a surface ECG, for example through delineation of the ECG in P-waves, QRS-complexes and/or T-waves, wherein each wave may be associated with specific atrial or ventricular activity.

Instable activation potentials may be such activation potentials that have an energy which is not well localized, have baseline deviations and/or large wave-to-wave amplitude deviations. These instable activation potentials may be excluded from further analysis, in particular by blanking. The instable activation potentials can be classified based on expected waveforms of the surface ECG and/or the intracardiac electrogram 2. Alternatively, instable activation potentials can be identified in the classification routine 15.

Noisy activation potentials are such activation potentials where the noise spectrum overlaps the physiological spectrum in a relevant manner. Known methods for noise estimation can be used.

Here and preferably the weighted blanking is an application of different weights 28 to the electrogram 2 inside the respective time interval 26. Preferably the weights 28 are predetermined to comprise a section of complete removal of the respective time interval 26, for example a multiplication with zero, and at least one section of reducing the amplitude of the electrogram 2. The weighted blanking can be applied by other methods than multiplication. An example of weights 28 may be a gauss curve.

The weighted blanking can be parameterized, in particular regarding the weights and/or the length of the blanking, based on the classification of the interference signal that is to be blanked. This way it becomes possible to detect local activation potentials 12 that have a high probability of showing a clear signal even during atrial fibrillation thereby allowing the analysis of local activation potentials during atrial fibrillation.

As an example, a pacing artifact can be entirely suppressed from the onset and a defined period following the onset after which the blanking is reduced. The length and/or reduction may be specific to hardware or software filter settings used with the control system 3.

A QRS complex may be dynamically penalized estimated from the associated influence on the last few beats. The present approach allows to track a local activation potential 12 that gradually delays and overlaps the QRS timing, which happens often at the critical moment of isolation. Therefore, the present approach allows determining the cardiac isolation status during an isolation procedure and during atrial fibrillation without the necessity to cardiovert the patient to a normal sinus rhythm.

FIG. 2 shows another example for a blanking of a time interval 29, in this case relative to the CS-potentials 13. Here, a complete blanking of the signal in time intervals 29 may be performed by multiplication with zero.

The interference signal removal step, in particular by means of blanking, may be performed with regard to the identification step on the intracardiac electrogram 2, while the classification routine 15 may be based on an analysis of the local activation potentials 12 in the intracardiac electrogram 2 without or a different type of interference signal removal as used for the identification.

The control system 3 may further execute a quality control step. In the quality control step, the control system 3 removes local activation potentials 12 and/or time sections of electrogram 2 channels 11 and/or electrogram 2 channels 11 as such, based on a quality parameter. The quality parameter may be a noise parameter, preferably a root-mean-square ratio and/or based on a power-line interference detection and/or the noise parameter may be a peak-to-baseline ratio.

With regards to FIG. 1, the multi-channel electrogram 2 may comprise at least four, preferably at least six, more preferably at least eight channels 11. The multi-channel electrogram 2 may have been recorded by an ablation catheter 6. Other possibilities are a circular catheter 6 or a multi-spline catheter 6 for example. The multi-channel electrogram 2 may be a bipolar electrogram. In a preferred alternative, the multi-channel electrogram 2 is a unipolar electrogram.

According to another teaching, which is of equal importance, a control system 3 configured to perform the proposed method is proposed. All explanations given with regards to the proposed method are fully applicable. The control system 3 is configured to receive and/or measure the multi-channel electrogram 2. The control system 3 is preferably connectable to the ablation catheter 6.

1-15. (canceled)

16. A method for determining a cardiac isolation status of a measurement location in the presence of far field interference by analyzing a multi-channel intracardiac electrogram of the measurement location via a control system, wherein:
in an identification routine the control system applies an activation search algorithm to analysis windows of at least 400 ms in at least two different channels of the intracardiac electrogram;
the activation search algorithm identifies windows of local activation potentials inside of the analysis windows; and
in a classification routine the control system analyzes the local activation potentials to determine the cardiac isolation status of the measurement location.

17. The method according to claim 16, wherein the intracardiac electrogram was recorded during an atrial arrhythmia and/or wherein the measurement location is at least partly located inside an atrium or at the entrance of a pulmonary vein and/or wherein the measurement location is at least partly located inside a pulmonary vein.

18. The method according to claim 16, wherein in the classification routine the control system analyzes a morphology of the local activation potentials to determine the cardiac isolation status of the measurement location.

19. The method according to claim 18, wherein in the classification routine the control system classifies the local activation potentials into morphology groups and determines the cardiac isolation status based on the distribution of local activation potentials across the morphology groups.

20. The method according to claim 18, wherein in the classification routine the control system classifies the local activation potentials into morphology groups based on a number of characteristic peaks of the local activation potential.

21. The method according to claim 20, wherein the control system classifies a peak with at least a predetermined amplitude and/or with at least a predetermined slope and/or with at most a predetermined slope and/or with at least a predetermined minimum peak distance and/or with at most a predetermined maximum peak distance and/or with a minimum peak angle and or with a maximum peak angle as one of the number of characteristic peaks.

22. The method according to claim 20, wherein the morphology groups comprise a group for local activation potentials with a single characteristic peak and/or exactly two characteristic peaks and/or exactly three characteristic peaks and/or more than three characteristic peaks and/or at least two characteristic peaks separated by a predetermined time.

23. The method according to claim 16, wherein the method includes one or more of the following features:
the analysis windows have a width of at least 400 ms;
the analysis windows have a width of at least 800 ms;
the analysis windows have a width of at least 1.25 s;
the analysis windows have a width of at most 3 s;
the analysis windows have a width of at most 2 s; and
the analysis windows have a width of at most 1.75 s.

24. The method according to claim 23, wherein the method includes one or more of the following features:
the analysis windows are overlapping or non-overlapping sliding windows over a measurement time for each channel;
at least two local activation potentials are extracted per channel;
at least one local activation potential is extracted per analysis window;
the measurement time is at least 1 s;
the measurement time is at least 2.5 s; and
the measurement time is at least 10 s.

25. The method according to claim 16, wherein the control system carries out the identification routine and the classification routine on a multi-channel intracardiac electrogram of the measurement location recorded after an ablation procedure applied near to the measurement location to determine the cardiac isolation status of the measurement location.

26. The method according to claim 25, wherein the control system carries out at least the identification routine on a multi-channel intracardiac electrogram of the measurement location recorded prior to the ablation procedure and determines the cardiac isolation status based on a comparison of the local activation potentials prior to and after the ablation procedure.

27. The method according to claim 16, wherein the activation search algorithm comprises a peak detection algorithm for finding the local activation potentials and subsequently the windows of the local activation potentials.

28. The method according to claim 27, wherein the method includes one or more of the following features:
the peak detection algorithm is based on non-linear filters;
the peak detection algorithm is based on wavelet filters;
the peak detection algorithm is based on a transformation of the electrogram;
the peak detection algorithm is based on a wavelet transformation; and
the peak detection algorithm comprises a peak detection by amplitude.

29. The method according to claim 16, wherein in the identification routine the control system identifies a fixed number of local activation potentials per channel and/or per analysis window.

30. The method according to claim 29, wherein the method includes one or more of the following features:
in the identification routine the control system identifies the fixed number of local activation potentials per time interval;
in the identification routine the control system identifies the fixed number of local activation potentials per time interval per measurement window;
the fixed number is based on a physiological and/or measured heart rate;
the fixed number is a maximum of one local activation potential per at least 500 ms;
the fixed number is a maximum of one local activation potential per at least 800 ms; and
the fixed number is a maximum of one local activation potential per at least 1 s.

31. The method according to claim 16, wherein the control system executes an interference signal removal step prior to the identification step, and wherein the interference signal removal step comprises complete or weighted blanking of time intervals around and/or relative to pacing artefacts and/or CS-potentials and/or ECG-waves.

32. The method according to claim 31, wherein the pacing artefacts and/or CS-potentials and/or ECG-waves are detected on an electrogram different from the multi-channel intracardiac electrogram, and wherein the electrogram different from the multi-channel intracardiac electrogram is a coronary sinus or surface electrogram.

33. The method according to claim 31, wherein the weighted blanking is an application of different weights to the electrogram inside the respective time interval, wherein the weights are predetermined to comprise a section of complete removal of the respective time interval and at least one section of reducing the amplitude of the electrogram.

34. The method according to claim 16, wherein the control system executes a quality control step, wherein in the quality control step the control system removes local activation potentials and/or time sections of electrogram channels and/or electrogram channels based on a quality parameter.

35. A control system configured to perform the method according to claim 16, wherein the control system is configured to receive and/or measure the multi-channel electrogram.
